(19) United States
(12) Patent Application Publication
(10) Pub. No.: US 2008/0138467 A1
(43) Pub. Date: Jun. 12, 2008

(54) Method For Producing Y-Aminobutyric-Acid-Containing Food And Yeast Having High Ability To Produce Y-Aminobutric Acid

(75) Inventors: Gyoufu Kaku, Chiba (JP); Toshihiko Hagiwara, Chiba (JP)

Correspondence Address: SUGHRUE MION, PLLC, 2100 PENNSYLVANIA AVENUE, N.W., SUITE 800, WASHINGTON, DC, 20037, US

(73) Assignee: Nichirei Foods Inc., Chuo-ku, Tokyo (JP)

(21) Appl. No.: 11/667,965

(22) PCT Filed: Nov. 10, 2005
(86) PCT No.: PCT/JP2005/020623

§ 371 Date: May 17, 2007

(30) Foreign Application Priority Data
Nov. 17, 2004 (JP) 2004-333671

(51) Int. Cl. A23L 1/28; C12N 1/16

(52) U.S. Cl. 426/62; 426/60; 435/255.5

(57) ABSTRACT

It is an objective of the present invention to provide a means of easily mass-producing γ-aminobutyric acid with the use of microorganisms.

The present invention relates to a method for producing a γ-aminobutyric-acid-containing food, comprising causing a yeast or a treated product thereof, which has the ability to produce γ-aminobutyric acid in the presence of a sugar or a metabolic intermediate of sugar metabolism through a fermentation reaction, to act on a sugar, a metabolic intermediate of sugar metabolism, both a sugar or a metabolic intermediate of sugar metabolism and a glutamic acid or a salt thereof.

### TECHNICAL FIELD

The present invention relates to a method for producing a γ-aminobutyric-acid-containing food and a yeast having high ability to produce the γ-aminobutyric acid that is used for such method.

### BACKGROUND ART

γ-aminobutyric acid (hereafter to be abbreviated as “GABA” in some cases) is a kind of nonprotein composition amino acid widely found in nature. As a food component, a minute amount of γ-aminobutyric acid is contained in various types of grains, vegetables, fruits, and mushrooms. Also, γ-aminobutyric acid is found in animal brains and spinal cords, and is thus known as a typical inhibitory neurotransmitter in mammal central nervous systems.

A wide range of physiological functions of γ-aminobutyric acid are known. Examples thereof include a function as a neurotransmitter, as described above, an antihypertensive function, a neuroleptic function, a renal-function-activating function, a liver-function-improving function, an antiobesity function, and an alcohol-metabolism-promoting function. In addition, since γ-aminobutyric acid has a function of improving cerebral blood flow and increasing oxygen supply so as to promote brain metabolism, it has been actually applied as a medicament to therapies for improving stroke sequelae and therapies for headache, tinnitus, depression, and the like caused by cerebral arteriosclerosis.

Thus, the intake of a sufficient amount of γ-aminobutyric acid in the daily diet when possible is believed to be useful for one's health maintenance and the prevention of various diseases. Therefore, a variety of methods for increasing the γ-aminobutyric acid content in foods by carrying out various types of physical or chemical treatment have been developed and reported. Known examples of such foods include “Gabaron tea” obtained by allowing tea leaves to be subjected to an anaerobic treatment in nitrogen gas (JP Patent Publication (Kokai) No. 63-103285 A (1988)), a GABA-enriched material, namely “oryza gaba,” obtained by allowing rice germ or rice bran containing germ to be subjected to an immersion treatment with water (JP Patent No. 2810993), GABA-containing “sprouted brown rice” obtained by allowing brown rice to sprout (JP Patent Publication (Kokai) No. 11-24694 A (1999)), and a GABA-rich “fermented Agaricus blazei extract” obtained through degradation of Agaricus enzyme.

Meanwhile, with the use of fermentation reaction functions and enzyme-degrading functions of naturally occurring microorganisms such as lactobacillus, yeast, and koji, many methods for producing γ-aminobutyric acid-rich food materials have been studied and reported. For instance, methods using lactobacillus (Patent Documents 1 to 8), a method using yeast (Patent Document 9), and methods using koji (Patent Documents 10 and 11) are known to the public.

However, it is difficult to say that the γ-aminobutyric acid contents of foods obtained by carrying out the physical or chemical treatments described above have reached a sufficient level to meet market demand. In addition, since such treated products are directly consumed as final food products, there is a limitation to the ability to add such products serving as functional materials to other foods, resulting in lack of the versatility. Further, in accordance with a method for mass-producing γ-aminobutyric acid through a fermentation reaction caused by lactobacillus, since γ-aminobutyric acid is produced as a product obtained through a simple enzyme reaction caused by glutamic acid decarboxylase that is contained in lactobacillus, functional components other than γ-aminobutyric acid are less likely to be obtained. Thus, it cannot be expected that further systematic functional effects will be exerted thereby. Furthermore, in accordance with a method for producing γ-aminobutyric acid by carrying out microbial treatments with the use of yeast, koji, and the like, desirable effects of producing γ-aminobutyric acid cannot be obtained without the use of, for example, yeasts that have been preliminary subjected to an acetone treatment so as to be dehydrated. Thus, treatment steps become complicated so as not to be appropriate for mass production in factories, resulting in cost increases. Even in cases in which yeasts subjected to an acetone treatment are used, unless yeasts (in the form of a dehydrated product) are added to a reaction solution in a manner such that the yeast concentration becomes as high as approximately 30% to 40% relative to the total volume of the solution, γ-aminobutyric acid cannot be mass-produced. Accordingly, it must be said that these methods lack practical usefulness.

[Patent Document 1] JP Patent Publication (Kokai) No. 6-45141 A (1994)

[Patent Document 2] JP Patent Publication (Kokai) No. 10-295394 A (1998)

[Patent Document 3] JP Patent Publication (Kokai) No. 2000-308457 A

[Patent Document 4] JP Patent Publication (Kokai) No. 2000-210075 A

[Patent Document 5] JP Patent Publication (Kokai) No. 2001-120179 A

[Patent Document 6] JP Patent Publication (Kokai) No. 2003-180389 A

[Patent Document 7] JP Patent Publication (Kokai) No. 2003-70462 A

[Patent Document 8] JP Patent No. 2704493

[Patent Document 9] JP Patent Publication (Kokai) No. 9-238650 A (1997)

[Patent Document 10] JP Patent Publication (Kokai) No. 10-165191 A (1998)

[Patent Document 11] JP Patent Publication (Kokai) No. 11-103825 A (1999)

### DISCLOSURE OF THE INVENTION

It is an objective of the present invention to provide a means of easily mass-producing γ-aminobutyric acid with the use of a microorganism. The present invention relates to the following (1) to (10).

(1) A method for producing a γ-aminobutyric-acid-containing food, comprising causing a yeast or a treated product thereof, which has the ability to produce γ-aminobutyric acid in the presence of a sugar or a metabolic intermediate of sugar metabolism through a fermentation reaction, to act on a sugar, a metabolic intermediate of sugar metabolism, or both a sugar or a metabolic intermediate of sugar metabolism and a glutamic acid or a salt thereof.
(2) The method described in (1), wherein the yeast is a yeast belonging to the genus Pichia or Candida.
(3) The method described in (1) or (2), wherein the yeast is Pichia anomala MR-1 (accession no. FERM BP-10134) or a mutant strain thereof having the ability to produce γ-aminobutyric acid.
(4) The method described in any one of (1) to (3), comprising causing the yeast or a treated product thereof to act on usable portions of animals, plants, or microorganisms, extracts from animals, plants, or microorganisms, or food materials made from the aforementioned usable portions or extracts, which contain a sugar, a metabolic intermediate of sugar metabolism, or both a sugar or a metabolic intermediate of sugar metabolism and a glutamic acid or a salt thereof.
(5) The method described in any one of (1) to (4), wherein a γ-aminobutyric acid production reaction is carried out under conditions in which the initial pH is 3.0 to 6.0 and that the temperature is 32° C. to 55° C.
(6) The method described in any one of (1) to (5), comprising the step of increasing the γ-aminobutyric acid concentration by allowing the reaction solution containing γ-aminobutyric acid to be further subjected to separation, purification, concentration, or dehydration.
(7) A γ-aminobutyric-acid-containing food, which is produced by the method according to any one of claims 1 to 6.
(8) A yeast belonging to Pichia anomala, which has the ability to produce γ-aminobutyric acid at a concentration of 150 mg/100 ml or higher upon measurement of the γ-aminobutyric acid concentration in a solution that is obtained in a manner such that: 1.0 g of viable cells (moisture content: 78.8% by weight) are added to a 200-ml Erlenmeyer flask that contains 50 ml of an aqueous solution containing glucose (5% by weight) and glutamic acid (1% by weight); the resultant is shaken at 45° C. for 24 hours, inactivated by heating at 85° C. for 15 minutes, and centrifuged; and the supernatant is concentrated so as to result in a constant volume of 25 ml.
(9) A yeast, which is Pichia anomala MR-1 (accession no. FERM BP-10134) or a mutant strain thereof having the ability to produce γ-aminobutyric acid.
(10) A food containing the yeast belonging to the genus Pichia.

### EFFECTS OF THE INVENTION

In accordance with the present invention, γ-aminobutyric acid can easily be mass-produced. Since the yeast of the present invention produces γ-aminobutyric acid through a fermentation reaction, γ-aminobutyric-acid-containing foods produced with the use of the yeast of the present invention contain useful fermentation products in addition to γ-aminobutyric acid.

This description includes part or all of the contents as disclosed in the description of Japanese Patent Application No. 2004-333671, which is a priority document of the present application.

### BRIEF DESCRIPTION OF THE DRAWINGS

FIG. 1 shows the results of comparing the MR-1 strain of the present invention and a known Pichia anomala yeast (AY231611.1) in terms of the nucleotide sequence of the ITS-5.8S rDNA gene. The homology therebetween was 97.6%.

FIG. 2 shows differences in influences of the sodium chloride concentrations upon the growth of the MR-1 yeast of the present invention and that of a known yeast NBRC-100267.

FIG. 3 shows influences of the culture temperatures upon the growth of the MR-1 yeast.

### BEST MODE FOR CARRYING OUT THE INVENTION

Hereafter, the present invention will be described in greater detail. In addition, the symbol “%” indicates “% by weight,” unless otherwise specified.

In order to enable the use of γ-aminobutyric acid as a functional component in a wider range of foods, the inventors of the present invention examined a method for easily mass-producing γ-aminobutyric acid with the use of a microorganism. As a result of intensive studies specifically directed to finding a microorganism in the form of a viable cell capable of producing γ-aminobutyric acid without being subjected to a particular treatment, they discovered a marine-derived yeast, which is a microorganism living in the ocean and having high ability to produce γ-aminobutyric acid through a reaction in a viable cell body in the presence of a sugar or a metabolic intermediate of sugar metabolism. With the use of such yeast, it was confirmed that it is possible to mass produce a naturally occurring metabolite such as γ-aminobutyric acid by a simple method in a short period of time. In addition, such yeast was confirmed to be a novel strain belonging to Pichia anomala based on genetic, physiological, and biochemical identification tests. The strain was designated as Pichia anomala MR-1 by the present inventors. The strain has been deposited with the International Patent Organism Depositary, National Institute of Advanced Industrial Science and Technology, at AIST (Tsukuba Central 6, 1-1-1 Higashi, Tsukuba, Ibaraki, Japan), as of Sep. 28, 2004 (under accession no. FERM BP-10134). The deposition was made by Nichirei Corporation, Processed Foods Company (9, Shinminato, Mihama-ku, Chiba-shi, Chiba, 261-8545, Japan) as of Sep. 28, 2004. As of Oct. 18, 2005, prior to the filing of the present application (date of receipt: Oct. 21, 2005), the strain was re-registered with Nichirei Foods Inc. named as the applicant of the present application (6-19-20 Tsukiji, Chuo-ku, Tokyo, 104-8402, Japan).

In accordance with the method for producing γ-aminobutyric acid or a food containing the same of the present invention, not only the strain Pichia anomala MR-1 but also a yeast having the ability to produce γ-aminobutyric acid in the presence of a sugar or a metabolic intermediate of sugar metabolism through a fermentation reaction is preferably used. A yeast having such ability is a yeast belonging to the genus Pichia or Candida. More specifically, examples thereof include, but are not limited to, Pichia anomala (e.g., Pichia anomala NBRC-10213 and Pichia anomala NBRC-100267), Pichia jadinii (anamorph: Candida utilis; e.g., Pichia jadinii NBRC-0987), and Candida utilis (e.g., Candida utilis NBRC-10707). Any of such yeasts can be used in the form of a suspension of yeast cells for the method of the present invention. Such yeast can be used in the form of a so-called immobilized yeast that is supported on an adequate carrier. Such immobilized yeast is an example of a “treated product” of the yeast used herein.

Hitherto, there have been no known yeasts that have the ability to produce γ-aminobutyric acid at a level equivalent to or exceeding the level of such ability imparted to the strain Pichia anomala MR-1. Examples of such yeasts include a yeast that has the ability to produce γ-aminobutyric acid at a concentration of 150 mg/100 ml or higher, preferably 200 gm/100 ml, and more preferably 300 mg/100 ml upon measurement of the γ-aminobutyric acid concentration in a solution obtained in a manner whereby: 1.0 g of viable cells (moisture content: 78.8% by weight) are added to a 200-ml Erlenmeyer flask that contains 50 ml of an aqueous solution containing glucose (5% by weight) and glutamic acid (1% by weight); the resultant is shaken at 45° C. for 24 hours, inactivated by heating at 85° C. for 15 minutes, and centrifuged; and the supernatant is concentrated so as to result in a constant volume of 25 ml. Also, a mutant strain of Pichia anomala MR-1 is preferably used, as long as such mutant has the ability to produce γ-aminobutyric acid. A mutagenic treatment may be carried out with the use of any adequate mutagen. Herein, the term “mutagen” in a broad sense should be understood to pertain to, for example, treatments inducing mutagenic effects such as UV irradiation, as well as drugs having mutagenic effects. Examples of an adequate mutagen include ethyl methanesulfonate, UV irradiation, N-methyl-N′-nitro-N-nitrosoguanidine, nucleotide base analogs such as bromouracil, and acridines. Also, any other effective mutagens can be used.

Pichia anomala MR-1 produces GABA in the presence of a sugar or a metabolic intermediate of sugar metabolism. Meanwhile, Pichia anomala MR-1 produces only a small amount of GABA in the presence of glutamic acid with the absence of a sugar or a metabolic intermediate of sugar metabolism. Pichia anomala MR-1 significantly differs from known GABA-producing yeasts that cannot produce GABA without the presence of glutamic acid or a salt thereof. In addition, it is remarkable that Pichia anomala MR-1 can produce a large amount of GABA in a synergistic manner in the presence of a sugar or a metabolic intermediate of sugar metabolism and glutamic acid or a salt thereof.

A GABA production reaction caused by Pichia anomala MR-1 is characterized in the following (1) to (5):

(1) a considerable amount of GABA is produced merely with the addition of a sugar or a metabolic intermediate of sugar metabolism even in the substantial absence of glutamic acid;

(2) other components such as free alanine (to be referred to as “Ala” in some cases herein) are also produced in the presence of a sugar or a metabolic intermediate of sugar metabolism, in addition to the production of a considerable amount of GABA;

(3) GABA is almost never produced in the absence of a sugar or a metabolic intermediate of sugar metabolism even with the addition of glutamic acid;

(4) a considerable amount of an ethanol component is detected in a GABA-containing reaction solution obtained by a GABA production reaction carried out in the presence of a sugar or a metabolic intermediate of sugar metabolism; and

(5) the amount of GABA produced decreases by approximately 50% or more than 50% in a case in which cells are used in a GABA production reaction after having been stored in a frozen state for two or more days, compared with a case in which cells are used after having been stored at a low temperature at which such cells remained unfrozen for the same number of days.

Given that GABA production caused by Pichia anomala MR-1 is based on a simple enzyme reaction with the use of a specific enzyme (as with the case of GABA production caused by lactobacillus), regardless of whether cells are dead or viable, there would be no significant difference in terms of the amount of GABA produced unless the enzyme involved in such reaction becomes inactivated. However, the phenomenon described in (5) above cannot be explained based on such hypothesis. Thus, it is assumed that GABA production caused by Pichia anomala MR-1 results from a kind of fermentation induced by a combination of intracellular metabolic functions. This assumption is supported by the fact that Ala and ethanol are simultaneously produced as described in (2) and (4) above. Therefore, when GABA is produced by the method of the present invention, it is expected that other useful components can be simultaneously produced. In addition, GABA production that is caused by Pichia anomala MR-1 does not require any preliminary treatment, such as an acetone treatment that is carried out in cases in which the usual yeasts are used. Also, such production is advantageous in that viable cells can be directly used. As shown in Test example 9, viable cells of the yeast of the genus Pichia or Candida have the very high ability to produce GABA, such ability being 5 to 15 times higher than that of other yeasts. Therefore, it is supposed that GABA production caused by the other yeast strains used in the present invention such as Pichia anomala NBRC-10213, Pichia anomala NBRC-100267, Pichia jadinii NBRC-0987, and Candida utilis NBRC-10707, is also based on a fermentation reaction.

In accordance with the present invention, examples of sugars that can be used for GABA production include monosaccharides, disaccharides, sugar alcohols, and oligosaccharides. Examples of monosaccharides include fructose, glucose, xylose, sorbose, and galactose. Examples of disaccharides include maltose, lactose, trehalose, sucrose, isomerized lactose, and palatinose. Examples of sugar alcohols include maltitol, xylitol, sorbitol, mannitol, and palatinit. Among them, glucose, fructose, maltose, and sucrose are preferable.

In accordance with the present invention, the term “metabolic intermediate of sugar metabolism” indicates each component of the sugar metabolic pathway, including the glycolytic pathway and the TCA cycle. Specific examples of such components include: glycolytic pathway components such as glycogen, various types of phosphorylated glucoses and degradation products thereof, and pyruvic acid; and TCA cycle components such as citric acid, isocitric acid, ketoglutaric acid, succinic acid, fumaric acid, malic acid, and oxaloacetic acid. In view of stability, practical usefulness as a starting material, and the like, glycogen, citric acid, pyruvic acid, ketoglutaric acid, succinic acid, and malic acid are preferable. In addition, since a metabolic intermediate of sugar metabolism can be a substrate for a GABA production reaction, it is understood that the GABA production reaction of the present invention is based on a fermentation reaction.

In addition, glutamic acid may be in the form of a salt such as sodium glutamate.

The amounts of a sugar or a metabolic intermediate of sugar metabolism and glutamic acid added are not particularly limited. For instance, in a case in which the yeast of the present invention (moisture content: 78%) exists at a concentration of 2% by weight in a reaction system, the concentration of glucose used as a sugar is preferably 1.0% to 10.0% by weight. For instance, in a case in which Pichia anomala MR-1 exists at a concentration of 2% by weight in a reaction system, the sufficient amount of glucose added is not less than 1% by weight in Test example 1 (with the absence of glutamic acid). In addition, in Test example 2 (with the presence of glutamic acid), the optimum amount of glucose added was 7.5% by weight. In such cases, the amounts of GABA produced increased approximately 8 and 100 times, respectively, compared with the amounts obtained under the glucose-free condition. Further, glutamic acid or a salt thereof is added at a concentration of preferably approximately 0.25% to 2.0% by weight and more preferably 0.5% to 1.5% by weight, for example, in a case in which the yeast of the present invention (moisture content: 78%) exists at a concentration of 2% by weight in a reaction system.

In a GABA production reaction, the initial pH upon the initiation of a reaction is preferably 3.0 to 6.0 and more preferably 4.0 to 5.0.

The optimum range of the reaction temperature can readily be determined by examining the relationship between the reaction temperature and the amount of GABA produced for each substrate to be used. Typically, the temperature range that is selected is between 32° C. and 55° C., preferably between 40° C. and 50° C., and more preferably between 43° C. and 48° C. As shown in Test example 4, a considerable amount of GABA is produced in the above temperature range. In addition, the present inventors confirmed that cell growth of the MR-1 strain does not substantially take place under the above temperature conditions in which a considerable amount of GABA is produced (see Test example 4). Thus, it can be said that a GABA production reaction caused by the MR-1 strain is characterized in that the reaction progresses under conditions in which cell growth is less likely to take place.

In general, the amount of cells added may be within the range of 2% to 10% by weight relative to the weight of the reaction solution (in a case in which cells having a moisture content of 78% are used). Considering the amount of GABA produced and the starting material costs in a comprehensive manner, the range of such amount is preferably 2% to 5% by weight (in a case in which the same cells are used).

Also, as with the case of reaction temperature, the optimum range of reaction time can readily be determined by examining the relationship between the reaction time and the amount of GABA produced for each substrate to be used. Typically, such reaction time is 12 to 72 hours. In particular, in a case in which the reaction temperature is 35° C. to 40° C., the sufficient reaction time is considered to be 48 to 72 hours. Also, in a case in which the reaction temperature is 40° C. to 50° C., the sufficient reaction time is considered to be approximately 12 to 48 hours.

For instance, in the case of a reaction system consisting of the strain Pichia anomala MR-1, a sugar or a metabolic intermediate of sugar metabolism, and glutamic acid, the adequate reaction time is within 24 hours at 45° C. and within 72 hours at 37° C. In a case in which components such as a temperature-sensitive component and a component that is easily discolored at high temperature are contained in such system, the reaction is preferably carried out at low temperature for a long period of time in some cases.

The aforementioned GABA production reaction may be carried out in any manner involving a batch-type method, a semicontinuous-type method, a continuous-type method, or the like.

Each component that serves as a reactive substrate in the present invention, such as a sugar, a metabolic intermediate of sugar metabolism, or both a sugar or a metabolic intermediate of sugar metabolism and glutamic acid or a salt thereof, is provided directly or in the form of a solution in an adequate solvent such as water (i.e., a reaction solution).

Such reactive substrates may be provided as food materials containing a sugar, a metabolic intermediate of sugar metabolism, or both a sugar or a metabolic intermediate of sugar metabolism and a glutamic acid or a salt thereof. Examples of such food materials include: usable portions of animals, plants, or microorganisms; extracts from animals, plants, or microorganisms; and food materials made from the aforementioned usable portions or extracts. In accordance with the present invention, the term “usable portions” indicates portions of animals, plants, or microorganisms that can be used as foods or products obtained through adequate treatments (e.g., pulverization, heating, baking, frying, dehydration, and braising).

If a food material originally contains a sugar, a metabolic intermediate of sugar metabolism, and/or glutamic acid or a salt thereof, it is not necessary to add these components. Thus, such food material can be directly used as a reactive substrate in the present invention. For instance, various types of fermented flavorings obtained by using a commercially available yeast extract, soybean, or wheat as a starting material often contain sufficient amounts of a sugar or a metabolic intermediate of sugar metabolism and glutamic acid. In addition, natural flavorings such as a seaweed extract and fish sauce are rich in glutamic acid derived from their starting materials. However, in some cases, such natural flavorings contain an unexpectedly low amount of a sugar or a metabolic intermediate of sugar metabolism. In such cases, considering the solid product content and the glutamic acid content in such food material, it is possible to use a product to which an adequate amount of a sugar or a metabolic intermediate of sugar metabolism such as glucose has been added (e.g., a system 5% of which is MR-1 yeast cells (moisture content: 78% by weight), to which sugar is then added such that the system contains 1% to 5% sugar) as a reactive substrate for the present invention. In addition, with the addition of a food enzyme such as amylase or protease to a food material, components (e.g., mainly starch and protein) in the food material are degraded. Thus, it becomes possible to use the food material, which has increased contents of a sugar, a metabolic intermediate of sugar metabolism, or glutamic acid, all of which have a relatively small molecular weight, as a reactive substrate for the present invention.

A reaction solution containing γ-aminobutyric acid that has been obtained by causing the yeast as defined above to react with the substrate as defined above may be directly added to various types of beverages or foods for use. Further, it is also possible to use such reaction solution after the γ-aminobutyric acid content has been increased by conventional food-processing steps (filtration, concentration, dehydration, pulverization, and the like). Furthermore, it is also possible to form pulverized products of thus obtained foods into tablets for use. In addition, the “γ-aminobutyric-acid-containing foods” produced by the present invention include a product containing the increased content of γ-aminobutyric acid (e.g., γ-aminobutyric acid isolated by a conventional separation means such as ion exchange or chromatography).

A food produced by the present invention may contain yeast in the form of viable cells, dead cells, or disrupted cells. In addition, cells of the yeast and disrupted cells thereof may be removed from such food.

Also, the present invention relates to food containing yeast belonging to the genus Pichia (particularly Pichia anomala). In such case, the yeast may also be contained in the form of viable cells, dead cells, or disrupted cells. Also, in such case, cells of the yeast belonging to the genus Pichia preferably contain γ-aminobutyric acid. In accordance with such embodiment of the present invention, a novel use of a yeast of the genus Pichia is provided.

### EXAMPLES

The present invention is hereafter described in greater detail with reference to the following examples, although the technical scope of the present invention is not limited thereto.

### Reference Example 1

Pichia anomala MR-1 is a kind of marine yeasts isolated from seawater of Hachinohe offing, Japan, in accordance with following procedures.

[Separation from Seawater]

First, approximately 50 liters of seawater at a water depth of 5 meters was collected with the use of a sterile water sampler on a ship located offshore of Hachinohe (approximately 15 kilometers away from the seacoast of Aomori, Japan). The seawater was transported under a low temperature at which the seawater remained unfrozen. On the next day, the seawater was filtered with 0.45 μm sterile membrane filters. Microorganism cells which remained on the filters were suspended in 15 ml of sterile distilled water and were used as a sample containing the cells isolated from seawater for the following experiments.

Each 200 μl were taken out from the samples (15 ml) above and spread onto plates of the YPD agar culture medium, which were used to culture of salt-resistant yeast, containing 2% glucose, 2% peptone, 1% yeast extract, 3% sodium chloride salt, 2% agar and 0.01% chloramphenicol (w/v). Plates were incubated at 25° C. for 5 days respectively. The cell bodies were taken out from the yeast-like microbe colonies grown on plates of the YPD agar, and morphologic of cells were observed with an optical microscope of 1000 times. Each single colony which seemed to be a yeast-like microbe was selected respectively. Then, the cell bodies were taken out again from each single colony above with a platinum loop and were suspended in 1 ml of sterile distilled water, and streaked on plates of another new YPD agar. The plates were incubated at 25° C. for 3-5 days. Meanwhile, in order to completely clone these microbes on the colonies, this procedure of such isolate and culture was repeated at least 5 times. As the last result, 58 strains of the yeast-like microbes were isolated from 50 liters of seawater.

### [Growth Culture of Separated Cells]

Cells were collected from each colony that had been finally isolated. The cells of each colony were used to inoculate 200 ml of a YPD liquid medium (glucose: 2%; peptone: 2%; yeast extract powder: 1%; sodium chloride: 3%; KH₂PO₄: 0.05%; MgSO₄: 0.05%; and (NH₄)₂SO₄: 0.1%), followed by shake culture at 25° C. for 2 to 3 days. Next, each culture solution was aseptically centrifuged. The cells were washed with sterilized water, followed by another centrifugation. In addition, in order to sufficiently wash cells, the series of operations (centrifugation and washing) was repeated 2 to 3 times.

### [Comparison in Terms of the Ability to Produce γ-Aminobutyric Acid]

The yeast-like microorganism cells subjected to separation and washing described above (0.5 g each) were added to reaction solutions (25 ml each) containing 5% glucose and 1% glutamic acid, followed by nitrogen gas filling and a reaction at 37° C. for 3 days. The obtained reaction solutions were inactivated by heating at 85° C. for 15 minutes, followed by centrifugation. Each supernatant was adjusted to have a constant volume of 50 ml so as to be subjected to analysis of free amino acid content. Based on the analysis results, 3 out of 58 strains were found to be yeast-like microorganisms having the ability to produce a certain amount of GABA. Of these, 1 strain was found to have high ability to produce γ-aminobutyric acid. The yeast-like microorganism having high ability to produce γ-aminobutyric acid was temporarily designated as MR-1.

In addition, a fully automatic JLC-500/V amino acid analyzer (JEOL Ltd.) was used for the analysis of the contents of free amino acids, including a GABA component.

### (The Same Analyzer Will be Used Hereinafter)

### [Molecular Phylogeny Analysis]

A genomic DNA component was extracted from viable cells of the above MR-1 yeast-like microorganism having high ability to produce γ-aminobutyric acid in accordance with a conventional method. Then, the rDNA sequence of the ITS-5.8S region in a ribosome was analyzed (see SEQ ID NO: 1).

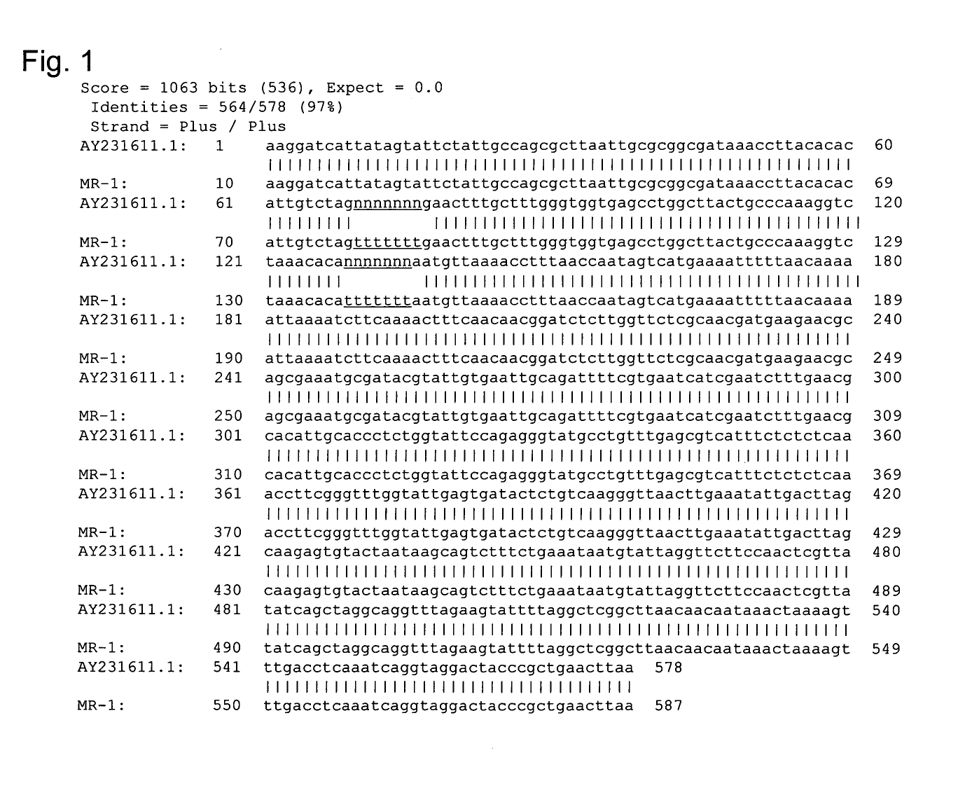

Regarding the nucleotide sequence of the ITS-5.8S rDNA gene (SEQ ID NO: 1), homology search was conducted based on the BLAST program by accessing the GenBank database. As a result, a 97.6% homology to a Pichia anomala yeast (AY231611.1) was confirmed (see FIG. 1).

In addition, based on molecular phylogeny analysis of the 18S rDNA nucleotide sequence, the strain of the present invention was found to have high homology to the Pichia anomala yeast (experimental data not shown).

Further, nucleotide sequences of yeast strains of the genus Pichia and those of representative yeast strains, which had been obtained from a DNA database, were subjected to multiple alignment, followed by homology search. Thus, the location of the strain of the present invention on the molecular phylogenetic tree was found to be identical to that of the Pichia anomala yeast.

### [Mycological, Physiological, and Biochemical Characteristics]

Table 1 shows mycological, physiological, and biochemical characteristics of the MR-1 strain described above.

TABLE 1
1. Mycological characteristics

| | |
|---|---|
| Morphology of cell | Globose, reproduction by budding |
| Morphology of colony | Circular, flattened, milky, viscous, and |
| | entirely smooth |
| Spore | With ascospores |
| Temperatures range of growth | 5° C. to 40° |
| pH range of growth | pH 3.0 to 8.0 |
| Aerobic/Anaerobic | Facultative anaerobe |

2. Physiological and biochemical characteristics
Fermentation of various carbohydrates

| | | | | | |
|---|---|---|---|---|---|
| D-Glucose | + | α.α- | − | Raffinose | + |
| | | Trehalose | | | |
| D-Galactose | − | Melibiose | − | Inulin | − |
| Maltose | + | Lactose | − | Soluble Starch | − |
| Methyl-α-D- | − | Cellobiose | − | D-Xylose | − |
| Glucopyranoside | | | | | |
| Sucrose | + | Melezitose | − | | |

Assimilation of various carbon sources

| | | | | | |
|---|---|---|---|---|---|
| D-Glucose | + | Arbutin | + | myo-Inositol | − |
| D-Galactose | + D | Melibiose | − | D-Glucono-1.5- | + |
| | | | | lactone | |
| L-Sorbose | − | Lactose | − | D-Gluconate | + |
| D-Glucosamine | − | Raffinose | + | D-Glucuronate | − |
| D-Ribose | + | Melezitose | + | D-Galacturonate | − |
| D-Xylose | + D | Inulin | − | DL-Lactate | + |
| L-Arabinose | − | Soluble | + | Succinate | + |
| | | Starch | | | |
| D-Arabinose | − | Glycerol | + | Citrate | + |
| L-Rhamnose | − | Erythritol | + | Methanol | − |
| Sucrose | + | Ribitol | + D | Ethanol | + |
| Maltose | + | Xylitol | + | Propane 1.2-diol | + D |
| α·α-Trehalose | + | L-Arabinitol | − | Butane 2.3-diol | + D |
| Methyl-α-D- | + | D-Glucitol | + | Quinic Acid | + |
| Glucopyranoside | | | | | |
| Cellobiose | + | D-Mannitol | + | | |
| Salicin | + | Galactitol | − | | |

Assimilation of various nitrogen sources

| | | | | | |
|---|---|---|---|---|---|
| Nitrate | + | Cadaverine | + | Imidazole | − |
| Nitrite | + D | Creatine | + | D-Tryptophane | + |
| Ethylamine | + | Creatinine | + | | |
| L-lysine | + | Glucosamine | + | | |

Explanation of representative symbols:
+: positive
D: delayed more than 7 days
−: negative

Based on the mycological, physiological, and biochemical characteristics listed in table 1, classification and identification were carried out with reference to YEASTS: Characteristics and identification (2000). The MR-1 strain was found to belong to Pichia anomala.

In addition, the MR-1 strain was not completely identical to a known strain of Pichia anomala. For instance, as shown in table 2, the MR-1 strain differs from a known strain of Pichia anomala (NBRC-100267) in terms of several physiological and biochemical characteristics.

TABLE 2
Differences of physiological and biochemical characteristics between
the MR-1 yeast and the known yeast Pichia anomala NBRC-100267

| | | NBRC-100267 |
|---|---|---|
| | MR-1 yeast | yeast |
| Assimilation of various carbon sources | | |
| L-Arabinose | − | + |
| D-Arabinose | − | + |
| Lactose | − | + |
| Inulin | − | + |
| L-Arabinitol | − | + D |
| Fermentation of various carbohydrates | | |
| D-Galactose | − | + D |
| Soluble starch | − | + D |

* Representative symbols have the same meanings as those used in table 1.

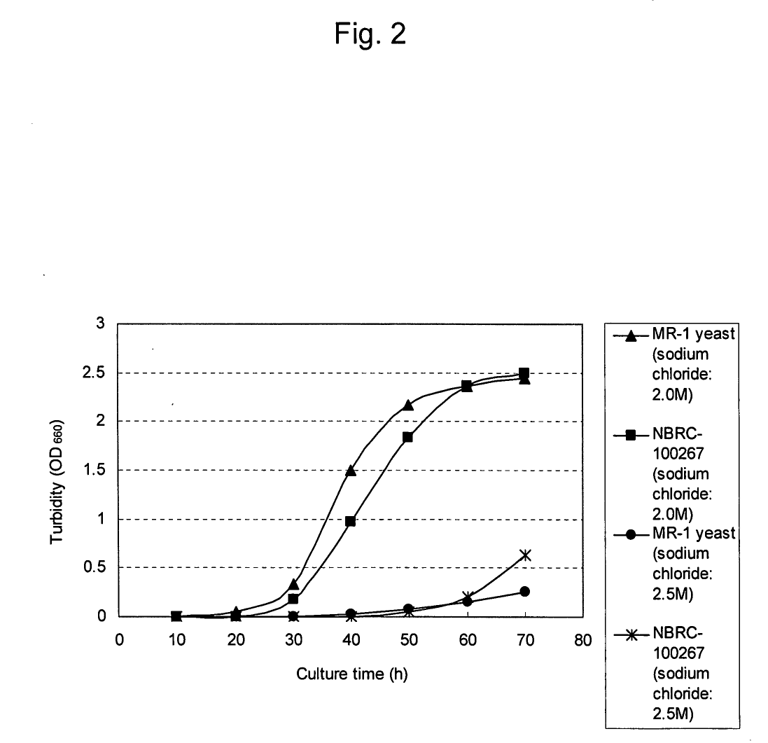

Further, the MR-1 strain differs from the known strain of Pichia anomala based on comparison in terms of growth rates at different salt concentrations (FIG. 2). Thus, it can be said that the MR-1 strain is a novel strain.

The yeast of the present invention has been deposited with the International Patent Organism Depositary, National Institute of Advanced Industrial Science and Technology, at AIST as Pichia anomala MR-1 (accession no. FERM BP-10134).

### Reference Example 2

### Reconstitution of the Preserved Strain

The MR-1 strain that had been preserved in a frozen or freeze-dried state was thawed at room temperature. Cells were collected with the use of a platinum loop. Then, the cells were dispersed in approximately 1 ml of sterilized water so as to be subjected to streak inoculation on a YPD agar medium, followed by culture at 25° C. for 5 days. Accordingly, MR-1 cells were obtained in the form of a milky circular colony on the medium. Such cells can be directly used for preculture as described below. In addition, the strain on the agar medium can be used for preculture with the proviso that it has been refrigerated at 5° C. to 10° C. for a period of 2 months or less.

### [Preculture]

Cells were collected from the colony of the agar medium. The cells were used to inoculate a sterilized YPD liquid medium (200 ml) in an Erlenmeyer flask, followed by shake culture at 25° C. for 2 to 3 days. During the culture, 1 ml of the culture solution was taken therefrom for measurement of the cell turbidity at a wavelength of 660 nm. When the cell turbidity was found to be 2.0 or higher, the culture solution was used for the subsequent main culture.

### [Main Culture]

7 liters of a liquid medium (glucose: 2%; urea: 2%; yeast extract powder: 1.0%; sodium chloride: 0.5%; KH₂PO₄: 0.05%; MgSO₄: 0.05%; and ammonia sulfate: 0.1%) was placed in a 10-liter jar fermentor, followed by heat sterilization at 121° C. for 60 minutes. The temperature of the liquid medium was cooled down to 30° C. Then, the pH of the culture solution was adjusted to 5.0 with the use of dilute hydrochloric acid and dilute alkali. Thereafter, 200 ml of the above culture solution was aseptically added to the jar fermentor, followed by aeration culture for 2 days with agitation at 25° C. The culture solution was centrifuged. The cells were then sufficiently washed with sterilized water, followed by additional centrifugation. After centrifugation and washing were repeated twice as described above, 150 g of MR-1 cells (moisture content: 78.8%) were obtained. The cells were used as MR-1 yeast cells in the following Test examples and Examples.

### Test Example 1

1.0 g of MR-1 cells (concentration: 2%) obtained in Reference example 2 above were added to each of six 200-ml Erlenmeyer flasks to which glucose aqueous solutions at predetermined concentrations (50 ml each) had been separately added. Each aqueous solution was subjected to a reaction for 24 hours while being shaken at 45° C. Thereafter, the aqueous solution was inactivated by heating at 85° C. for 15 minutes, followed by centrifugation. Each supernatant was concentrated so as to have a constant volume of 25 ml, followed by analysis of free amino acids, including GABA. In addition, as a result of analysis of free amino acids, the concentrations of GABA and Ala were found to be higher than those of the other free amino acids. Thus, the concentrations of these two types of amino acids are shown in table 3. In addition, the glutamic acid concentration was very low. However, since the glutamic acid concentration is deeply involved in GABA production, it is also listed in table 3.

TABLE 3
Influence of addition concentration of glucose on GABA production

| | Glucose concentration (%) | | | | | |
|---|---|---|---|---|---|---|
| | 0 | 1 | 3 | 5 | 7.5 | 10 |
| GABA concentration | 1.7 | 14.7 | 15.0 | 14.7 | 14.3 | 13.4 |
| (mg/100 ml) | | | | | | |
| Alanine concentration | 2.4 | 16.0 | 16.5 | 16.3 | 16.4 | 15.7 |
| (mg/100 ml) | | | | | | |
| Glutamic acid concentration | 7.4 | 0.3 | 0.4 | 0.4 | 0.3 | 0.4 |
| (mg/100 ml) | | | | | | |

In the above results, GABA production and Ala production were barely observed in the case in which glucose had not been added. Meanwhile, in the cases in which glucose had been added, it was found that the amounts of GABA and Ala produced significantly increased.

### Test Example 2

A GABA production reaction was carried out in the same manner as that of Test example 1 except that glutamic acid (concentration: 1%) was added to each reaction solution. Thus, concentrates (25 ml each) were prepared. The GABA and Ala concentrations of these concentrates are shown in table 4. Further, as an index for a fermentation reaction, the ethanol concentration was also measured.

TABLE 4
Influence of addition concentration of glucose on GABA production
(with the addition of 1% glutamic acid)

| | Glucose concentration (%) | | | | | |
|---|---|---|---|---|---|---|
| | 0 | 1 | 3 | 5 | 7.5 | 10 |
| GABA concentration | 3.9 | 258.4 | 312.8 | 324.3 | 391.4 | 234.9 |
| (mg/100 ml) | | | | | | |
| Alanine concentration | 5.4 | 86.7 | 98.7 | 105.4 | 112.3 | 99.5 |
| (mg/100 ml) | | | | | | |
| Ethanol concentration | 0 | 285.6 | 419.2 | 442.4 | 631.2 | 972.0 |
| (mg/100 ml) | | | | | | |

In the above results, in the case in which glucose had not been added, the production of GABA and that of Ala were barely observed even with the addition of glutamic acid. Meanwhile, in systems to which glucose and glutamic acid had been added, the amounts of GABA and Ala produced significantly increased compared with Test example 1.

The reason for the lower likelihood of observing the production of GABA even with the addition of glutamic acid in the cases in which glucose had not been added was considered to be the lack of yeast fermentation reaction progress in the absence of sugars.

### Test Example 3

Citric acid-sodium acid phosphate buffers (0.1 M citric acid-0.2 M disodium hydrogen phosphate) at different pH levels from 3.0 to 7.0 were each adjusted to have a constant volume of 50 ml with addition of MR-1 cells (2%), glucose (5%), and glutamic acid (1%) obtained in Reference example 2 above. A GABA production reaction was carried out under the same conditions as those of Test example 1. Accordingly, the concentrates (25 ml each) were obtained. The GABA concentrations of these concentrates are shown in table 5.

TABLE 5
Influence of the pH of a reaction solution on GABA production

| | pH of reaction solution | | | | |
|---|---|---|---|---|---|
| | 3.0 | 4.0 | 5.0 | 6.0 | 7.0 |
| GABA concentration (mg/100 ml) | 128.2 | 141.8 | 276.6 | 112.2 | 8.1 |

Based on the above results, it was determined that considerable amounts of GABA are produced within a wide pH range (3.0 to 6.0). In addition, the optimum pH was approximately pH 5.0.

### Test Example 4

10 ml of a reaction solution containing MR-1 cells (2%), glucose (5%), and glutamic acid (1%) obtained in Reference example 2 above was added to each of 12 L-shaped 20-ml test tubes. Each tube was shaken at 20 rpm such that a reaction was carried out with a temperature gradient from 15° C. to 60° C. for 3 days. Thereafter, separation and purification were carried out in the same manner as Test example 1 such that concentrates (5 ml each) were prepared. The GABA concentrations of these concentrates are shown in table 6.

TABLE 6
Influence of reaction temperature on GABA production

| | Reaction temperature (° C.) | | | | | |
|---|---|---|---|---|---|---|
| | 15 | 19.6 | 23.3 | 26.6 | 30 | 33.1 |
| GABA concentration | 2.4 | 2.8 | 3.5 | 9.5 | 11.0 | 42.9 |
| (mg/100 ml) | | | | | | |
| | Reaction temperature (° C.) | | | | | |
| | 36 | 40.5 | 44 | 48 | 52.9 | 60 |
| GABA concentration | 76.5 | 99.8 | 229.8 | 187.0 | 70.6 | 28.3 |
| (mg/100 ml) | | | | | | |

Based on the above results, it was determined that relatively large amounts of GABA are produced within a temperature range of 32° C. to 55° C., with the optimum reaction temperature being around 44° C. In addition, since the GABA concentration at 44° C. was approximately 3 times as large as that at 36° C., the reaction temperature is assumed to significantly influence GABA production.

The results obtained above also support the conclusion that GABA production by MR-1 is carried out through a fermentation reaction. This conclusion is based on the following reasoning. The amount of GABA produced at 52.9° C. accounts for approximately 30% of that at 44° C. In addition, in another experiment carried out under the same conditions (data not shown), the amount of GABA produced at 50° C. accounts for approximately 18% of that at 45° C. In general, it is unlikely that there is such significant difference between enzyme activity at 50° C. and that at 45° C. Thus, it is considered that the difference in terms of the amount of GABA produced results from the fact that the number of viable cells decreases due to increased temperatures such that fermentation becomes less likely to be induced.

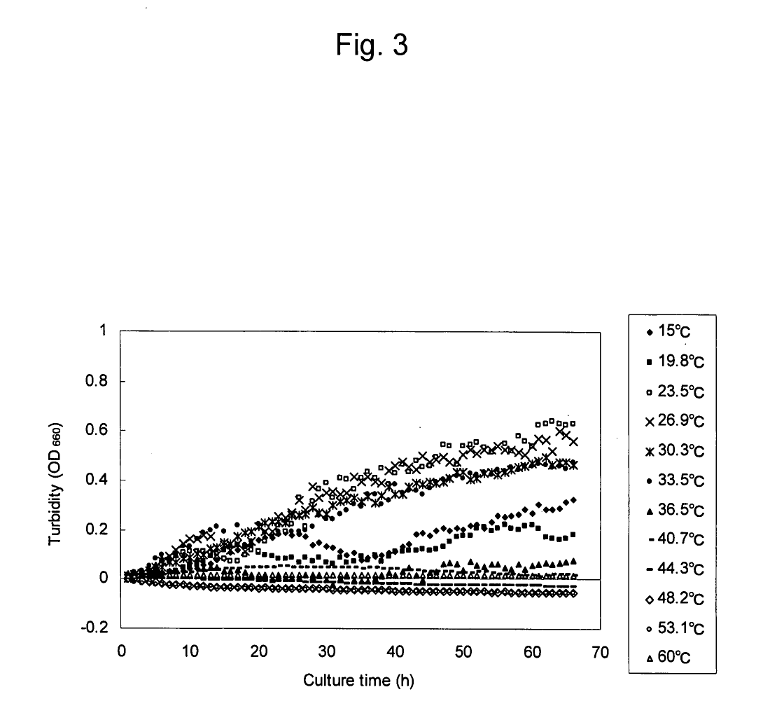

Further, the inventors of the present invention cultured MR-1 cells under the same conditions as those described above at temperatures of 15.0° C., 19.8° C., 23.5° C., 26.9° C., 30.3° C., 33.5° C., 36.5° C., 40.7° C., 44.3° C., 48.2° C., 53.1° C., and 60.0° C. Then, the turbidity (OD₆₆₀) of each culture solution was measured in a time-dependent manner such that the relationship between reaction temperatures and cell growth could be examined. Cell growth was not substantially observed at temperatures of 36.5° C. or higher (FIG. 3).

### Test Example 5

Glutamic acids at predetermined concentrations were separately added to reaction solutions (50 ml each) containing MR-1 cells (2%) and glucose (5%) obtained in reference example 2. A GABA production reaction was carried out under the same conditions as those of Test example 1. Accordingly, concentrates (25 ml each) were prepared. The GABA concentrations of these concentrates are shown in table 7.

TABLE 7
Influence of addition concentration of glutamic acid on
GABA production

| | Glutamic acid concentration (%) | | | | | |
|---|---|---|---|---|---|---|
| | 0.25 | 0.5 | 0.75 | 1.0 | 2.0 | 5.0 |
| GABA concentration | 204.2 | 300.3 | 339.4 | 347.7 | 287.4 | 150.0 |
| (mg/100 ml) | | | | | | |

Based on the above results, it was determined that an adequate concentration of glutamic acid added is in the range of approximately 0.25% to 2.0%, and more preferably 0.5% to 1.5%. On the other hand, in the cases in which the concentration of glutamic acid exceeded the above range, the amount of GABA produced tended to decrease.

### Test Example 6

MR-1 cells obtained in Reference example 2 at predetermined concentrations were separately added to reaction solutions (50 ml each) containing glucose (5%) and glutamic acid (1%). A GABA production reaction was carried out under the same conditions as those of Test example 1. Accordingly, concentrates (25 ml each) were prepared. The GABA concentrations of these concentrates are shown in table 8.

TABLE 8
Influence of addition concentration of MR-1 cells on
GABA production

| | MR-1 cell concentration (%) | | | | |
|---|---|---|---|---|---|
| | 1.0 | 2.0 | 3.0 | 5.0 | 10.0 |
| GABA concentration (mg/100 ml) | 119.3 | 331.8 | 327.1 | 323.9 | 305.8 |

Based on the above results, it was determined that the optimum concentration of MR-1 cells added is approximately 2% to 5%. In the cases of concentrations of MR-1 cells exceeded the above range, the amount of GABA produced tended not to increase.

### Test Example 7

Predetermined sugars (5% each) were separately added to reaction solutions (50 ml each) containing MR-1 cells (2%) and glutamic acid (1%) obtained in reference example 2. A GABA production reaction was carried out under the same conditions as those of Test example 1. Accordingly, concentrates (25 ml each) were prepared. The GABA concentrations of these concentrates are shown in table 9.

TABLE 9
Influence of kind of the adding saccharide on GABA production

| | Kind of saccharide | | | | |
|---|---|---|---|---|---|
| | Maltose | Sucrose | Lactose | Galactose | Sorbitol |
| GABA | 78.7 | 106.5 | 9.0 | 9.1 | 22.5 |
| concentration | | | | | |
| (mg/100 ml) | | | | | |
| | Kind of saccharide | | | | |
| | Fructose | Glucose | Xylose | Sorbose | Mannitol |
| GABA | 200.4 | 276.3 | 28.5 | 16.0 | 11.6 |
| concentration | | | | | |
| (mg/100 ml) | | | | | |

Based on the above results, it was determined that the amount of GABA produced tends to differ depending on the type of sugars added. Of these, monosaccharides such as glucose and fructose and disaccharides such as maltose and sucrose were found to have improved effects of promoting GABA production. In particular, glucose and fructose were found to have the maximal effects.

### Test Example 8

MR-1 cells that had been stored in an unfrozen state at 5° C. for 2 days and MR-1 cells that had been stored in a frozen state at −25° C. for 2 days at predetermined concentrations were separately added to reaction solutions (50 ml each) containing glucose (5%) and glutamic acid (1%). A GABA production reaction was carried out under the same conditions as those of Test example 1. The resulting concentrates (25 ml each) were prepared. In addition, the cells used were obtained in Reference example 2 above. The GABA concentrations of these concentrates are shown in table 10.

TABLE 10
Influence of the preservation state of MR-1 cells on GABA production

| | Cells stored in an | | Cells stored in a | |
|---|---|---|---|---|
| | unfrozen state | | frozen state | |
| MR-1 cell concentration (%) | 2.0 | 6.0 | 2.0 | 6.0 |
| GABA concentration (mg/100 ml) | 196.8 | 260.6 | 92.8 | 136.6 |

Based on the above results, it was determined that storage in a frozen state caused the cells to significantly deteriorate its ability to produce GABA. Thus, it is understood that it is preferable to use viable yeast cells in the present invention.

### Test Example 9

MR-1 cells (Reference example 2) of the present invention, other yeasts belonging to the genera Pichia or Candida (obtained from depository institutions), a commercially available marine-derived yeast (Yeast M, Sankyo Co., Ltd.), a bakers' yeast (Oriental Yeast Co., Ltd.), and Sake yeast kyokai No. 7 (1 g each) were separately added to reaction solutions (50 ml each) containing glucose (5%) and glutamic acid (1%). A GABA production reaction was carried out under the same conditions as those of Test example 1. Accordingly, concentrates (25 ml each) were prepared. The GABA concentrations of these concentrates are shown in table 11.

TABLE 11
Comparison of GABA production ability by various yeasts

| | GABA |
|---|---|
| | concentration |
| Yeast name | (mg/100 ml) |
| Pichia anomala MR-1 | 331.8 |
| Pichia anomala NBRC-10213 | 93.6 |
| Pichia anomala NBRC-100267 | 139.4 |
| Pihcia jadinii NBRC-0987 (Anamorph: Candida utilis) | 126.5 |
| Candida utilis NBRC-10717 | 143.2 |
| Saccharomyces cerevisiae (1) (Yeast M, Sankyo) | 24.0 |
| Saccharomyces cerevisiae (2) (Bakers' yeast) | 22.4 |
| Saccharomyces cerevisiae (3) (Sake yeast kyokai No. 7) | 17.3 |

Based on the above results, it was determined that the yeasts belonging to the genera Pichia and Candida have very high abilities to produce GABA, compared with other yeasts. In particular, the MR-1 yeasts of the present invention were found to have very high ability to produce GABA, at levels approximately 10 to 15 times greater than those of usual yeasts.

### Test Example 10

Glutamic acid-free reaction solutions and reaction solutions to which 1% glutamic acid had been added (50 ml each) were prepared. Both reaction solutions contained MR-1 cells (2%) (reference example 2) and predetermined 50 mM sugar or metabolic intermediates of sugar metabolism listed in the following table. A GABA production reaction was carried out under the same conditions as those of Test example 1. Accordingly, concentrates (25 ml each) were prepared. The GABA concentrations of these concentrates are shown in table 12.

TABLE 12
Influence of kind of adding metabolic intermediates of sugar
metabolism on GABA production

| | | Pyruvic | Ketoglutaric | Succinic | Malic |
|---|---|---|---|---|---|
| | Glucose | acid | acid | acid | acid |
| Glutamic-acid-free | 13.6 | 9.1 | 11.8 | 13.4 | 5.8 |
| 1% glutamic | 238.2 | 107.3 | 50.2 | 60.2 | 54.4 |
| acid-containing | | | | | |

Based on the above results, it was determined that GABA is produced from different metabolic intermediates of sugar metabolism in a glutamic acid-free system, and that GABA production is further promoted in a system to which glutamic acid (1%) has been added.

### Example 1

In accordance with the method for main culture in Reference example 2 above, 7 liters of a liquid medium was placed in a 10-liter jar fermentor, followed by sterilization. Then, 0.7 g of the MR-1 strain obtained in Reference example 2 was used to aseptically inoculate the liquid medium, followed by aeration culture at pH 5.0 and 30° C. for 2 days. The resulting culture solution was centrifuged and the cells were washed with sterilized water. Accordingly, 160 g of MR-1 cells (moisture content: 79.2%) were obtained. The cells were dispersed in 8 liters of a reaction solution containing glucose (3.0%) and glutamic acid (0.5%). The resultant was subjected to a GABA production reaction for 24 hours while being shaken at 45° C. Thereafter, the reaction solution was inactivated by heating at 85° C. for 15 minutes, followed by centrifugation. The supernatant was filtered and vacuum-concentrated. Thus, 800 ml of a concentrate with a solid content of 40% was obtained. In addition, analytical values for the content of GABA and other components in the concentrate are shown in table 13.

TABLE 13
Content of various useful components in the reaction solution of GABA
production by MR-1 strain

(unit: mg/100 ml)

| | Component | | | | | | | | |
|---|---|---|---|---|---|---|---|---|---|
| | | | | | | | | Malic | Succinic |
| | GABA | Glu | Ala | Gly | Lys | Ser | Pro | acid | acid |
| Content | 1576 | 2528 | 511.5 | 25.7 | 17.1 | 10.2 | 9.9 | 680.2 | 623.8 |

### Example 2

20 g of a commercially available “powdered fermented flavor enhancer S” (Kikkoman) was diluted 5-fold with distilled water. 5 g of MR-1 cells (concentration: 5%) of the present invention obtained in Reference example 2 were added thereto so as to be dispersed therein. The initial pH of the obtained reaction solution was adjusted to 5.0 with dilute hydrochloric acid. Then, a GABA production reaction was carried out at 45° C. for 24 hours. Thereafter, the reaction solution was inactivated by heating at 85° C. for 15 minutes, followed by centrifugation. The supernatant was concentrated such that 60 g of the concentrate was obtained. The GABA concentration in the concentrate was 171.2 mg/100 ml. Thus, compared with the GABA concentration of 4.1 mg/100 ml in a solution obtained by diluting an untreated starting material 3-fold, the GABA content was approximately 40 times as great.

### Example 3

100 g of a commercially available “Taimi JF” fish meat extract (SenmiEkisu Co., Ltd.) was diluted 3-fold with distilled water. 15 g of MR-1 cells (concentration: 5%) of the present invention obtained in Reference example 2 were added thereto so as to be dispersed therein. Then, a GABA production reaction was carried out in the same manner as that of Example 2 such that 100 g of the concentrate was obtained. The GABA concentration in the concentrate was 246.8 mg/100 ml. Thus, compared with the GABA concentration of 5.3 mg/100 ml in an untreated starting material, the GABA content was approximately 46 times as great.

### Example 4

100 g of a commercially available “dried bonito extract (Katsuo bushi ekisu) J” (SenmiEkisu Co., Ltd.) was diluted 3-fold with distilled water. 15 g of MR-1 cells (concentration: 5%) of the present invention obtained in Reference example 2, 15 g of glucose (concentration: 5%), and 3 g of sodium glutamate (concentration: 1%) were added thereto so as to be dispersed therein. Then, a GABA production reaction was carried out in the same manner as that of Example 2 such that 100 g of the concentrate was obtained. The GABA concentration in the concentrate was 306.3 mg/100 ml. Thus, compared with the GABA concentration in an untreated starting material (GABA-free), the amount of GABA produced was greater.

### Example 5

100 g of a commercially available “seaweed broth (Konbu dashi) KW-1” (Fuji Foods Corporation) was diluted 3-fold with distilled water. MR-1 cells obtained in Reference example 2, glucose, and sodium glutamate at predetermined concentrations shown in table 14 were added thereto so as to be dispersed therein. Then, a GABA production reaction was carried out in the same manner as that of Example 2 such that the concentrates (100 g each) were obtained. The GABA concentrations in the concentrates were examined. Compared with the GABA concentration in an untreated starting material (GABA-free), the amount of GABA produced was greater, even in the case in which the MR-1 cells of the present invention alone had been added. Further, in the case in which glucose and glutamic acid had been added in addition to the MR-1 cells, the GABA concentration was approximately 1.7 times as great.

TABLE 14
Comparison of GABA concentration in seaweed extracts
prepared by fermentation of MR-1 cells

| | | | (3) Starting |
|---|---|---|---|
| | | | material + 5% |
| | (1) Starting | (2) Starting | MR-1 cells + 5% |
| | material | material + 5% | glucose + 1% |
| | alone | MR-1 cells | glutamic acid |
| Amount of | 0 | 220.4 | 383.9 |
| GABA produced | | | |
| (mg/100 ml) | | | |

### Example 6

100 g of a commercially available livestock meat extract, namely “chicken meat extract C-501NAC” (Fuji Foods Corporation), was diluted 3-fold with distilled water. MR-1 cells obtained in Reference example 2, glucose, and sodium glutamate at predetermined concentrations shown in table 15 were added thereto so as to be dispersed therein. Then, a GABA production reaction was carried out in the same manner as that of Example 2 such that the concentrates (100 g each) were obtained. The GABA concentrations in the concentrates were examined. Compared with the GABA concentration in an untreated starting material (GABA-free), the amount of GABA produced was greater even in the case in which the MR-1 cells of the present invention alone had been added. Further, in the case in which glucose and glutamic acid had been added in addition to the MR-1 cells, the GABA concentration was at least twice as great.

TABLE 15
Comparison of GABA concentration in chicken extracts
prepared by fermentation of MR-1 cells

| | | | (3) Starting |
|---|---|---|---|
| | | | material + 5% |
| | (1) Starting | (2) Starting | MR-1 cells + 5% |
| | material | material + 5% | glucose + 1% |
| | alone | MR-1 cells | glutamic acid |
| GABA | 0 | 146.5 | 328.4 |
| concentration | | | |
| (mg/100 ml) | | | |

### Example 7

100 g of a commercially available livestock meat extract, namely “pork meat extract FP-301” (Fuji Foods Corporation), was diluted 3-fold with distilled water. MR-1 cells obtained in Reference example 2, glucose, and sodium glutamate at predetermined concentrations shown in table 16 were added thereto so as to be dispersed therein. Then, a GABA production reaction was carried out in the same manner as that of Example 2 such that the concentrates (100 g each) were obtained. The GABA concentrations in the concentrates were examined. Compared with the GABA concentration in an untreated starting material (GABA-free), the amount of GABA produced was greater, even in the case in which the MR-1 cells of the present invention had been added. Further, in the case in which glucose and glutamic acid had been added in addition to the MR-1 cells, the GABA concentration was approximately 4 times as great.

TABLE 16
Comparison of GABA concentration in pork extracts
prepared by fermentation of MR-1 cells

| | | | (3) Starting |
|---|---|---|---|
| | | | material + 5% |
| | (1) Starting | (2) Starting | MR-1 cells + 5% |
| | material | material + 5% | glucose + 1% |
| | alone | MR-1 cells | glutamic acid |
| GABA | 0 | 90.0 | 355.8 |
| concentration | | | |
| (mg/100 ml) | | | |

All publications, patents, and patent applications cited herein are incorporated herein by reference in their entirety.

1. A method for producing a γ-aminobutyric-acid-containing food, comprising causing a yeast or a treated product thereof to act on a sugar, a metabolic intermediate of sugar metabolism, both a sugar and glutamic acid, both a metabolic intermediate of sugar and glutamic acid, both a sugar and a salt of glutamic acid, or both a metabolic intermediate of sugar and a salt of glutamic acid, wherein
both the yeast and the treated product thereof have the ability to produce γ-aminobutyric acid in the presence of a sugar or a metabolic intermediate of sugar metabolism through a fermentation reaction.

2. The method according to claim 1, wherein the yeast is a yeast belonging to the genus Pichia or Candida.

3. The method according to claim 1, wherein the yeast is Pichia anomala MR-1 (accession no. FERM BP-10134) or a mutant strain thereof having the ability to produce γ-aminobutyric acid.

4. The method according to claim 1, comprising causing the yeast or a treated product thereof to act on usable portions of animals, plants, or microorganisms, extracts from animals, plants, or microorganisms, or food materials made from the aforementioned usable portions or extracts, which contain a sugar, a metabolic intermediate of sugar metabolism, both a sugar and glutamic acid, both a metabolic intermediate of sugar and glutamic acid, both a sugar and a salt of glutamic acid, or both a metabolic intermediate of sugar and a salt of glutamic acid.

5. The method according to claim 1, wherein a γ-aminobutyric acid production reaction is carried out under conditions in which the initial pH is 3.0 to 6.0 and that the temperature is 32° C. to 55° C.

6. The method according to claim 1, comprising the step of increasing the γ-aminobutyric acid concentration by allowing the reaction solution containing γ-aminobutyric acid to be further subjected to separation, purification, concentration, or dehydration.

7. A γ-aminobutyric-acid-containing food, which is produced by the method according to claim 1.

8. A yeast belonging to Pichia anomala, which has the ability to produce γ-aminobutyric acid at a concentration of 150 mg/100 ml or higher upon measurement of the γ-aminobutyric acid concentration in a solution that is obtained in a manner such that: 1.0 g of viable cells (moisture content: 78.8% by weight) are added to a 200-ml Erlenmeyer flask that contains 50 ml of an aqueous solution containing glucose (5% by weight) and glutamic acid (1% by weight); the resultant is shaken at 45° C. for 24 hours, inactivated by heating at 85° C. for 15 minutes, and centrifuged; and the supernatant is concentrated so as to result in a constant volume of 25 ml.

9. A yeast, which is Pichia anomala MR-1 (accession no. FERM BP-10134) or a mutant strain thereof having the ability to produce 7-aminobutyric acid.

10. A food containing a yeast belonging to the genus Pichia.


SEQUENCE LISTING (1 sequence)

SEQ ID NO: 1 - DNA, 578 bp, Pichia anomala
aaggatcattatagtattctattgccagcgcttaattgcgcggcgataaaccttacacacattgtctagtttttttgaactttgctttgggtggtgagcctggcttactgcccaaaggtctaaacacatttttttaatgttaaaacctttaaccaatagtcatgaaaatttttaacaaaaattaaaatcttcaaaactttcaacaacggatctcttggttctcgcaacgatgaagaacgcagcgaaatgcgatacgtattgtgaattgcagattttcgtgaatcatcgaatctttgaacgcacattgcaccctctggtattccagagggtatgcctgtttgagcgtcatttctctctcaaaccttcgggtttggtattgagtgatactctgtcaagggttaacttgaaatattgacttagcaagagtgtactaataagcagtctttctgaaataatgtattaggttcttccaactcgttatatcagctaggcaggtttagaagtattttaggctcggcttaacaacaataaactaaaagtttgacctcaaatcaggtaggactacccgctgaacttaa